(19) United States
(12) Patent Application Publication
(10) Pub. No.: US 2009/0087444 A1
(43) Pub. Date: Apr. 2, 2009

(54) PHARMACEUTICAL COMPOSITION COMPRISING POLYSACCHARIDES FROM ANGELICA GIGAS NAKAI FOR ACTIVATION OF DENDRITIC CELLS

(76) Inventors: Hwan-Mook Kim, Daejeon (KR); Sang-Bae Han, Cheongju-si (KR); Song-Kyu Park, Daejeon (KR); Ki-Ho Lee, Seoul (KR); Jong-Soon Kang, Daejeon (KR); Ki-Hoon Lee, Daejeon (KR); Jee-Youn Kim, Daejeon (KR); Chang-Woo Lee, Daejeon (KR)

Correspondence Address: JONES DAY, 222 EAST 41ST ST, NEW YORK, NY, 10017, US

(21) Appl. No.: 12/298,903

(22) PCT Filed: Sep. 29, 2006
(86) PCT No.: PCT/KR2006/003926

§ 371 Date: Oct. 28, 2008

(30) Foreign Application Priority Data
Jun. 2, 2006 (KR) 10-2006-0049937

(51) Int. Cl. A61K 39/00

(52) U.S. Cl. 424/184.1

(57) ABSTRACT

The present invention relates to a pharmaceutical composition for activating dendritic cells having polysaccharides from Angelica gigas Nakai as valid component. The angelan, polysaccharides separated from Angelica gigas Nakai, can improve the revelation of surface molecule of dendritic cells, the secret of cytokine of dendritic cells, the proliferation capability of T cells of dendritic cells, and the generation of cytokine such as IL-2.

### TECHNICAL FIELD

The present invention relates to a pharmaceutical composition for activating dendritic cells having polysaccharides from Angelica gigas Nakai as valid component. More particularly, the present invention relates to the use of immunomodulating drug that increases the maturity of dendritic cells, where the dendritic cells make the surface molecule of dendritic cells of angelan which is polysaccharides divided from Angelica gigas Nakai to be revealed, make cytokine of dendritic cells to be secreted, increase the ability of proliferating T cell of dendritic cells, and increase the generation of cytokine of IL-2 from T cell.

### BACKGROUND ART

Dendritic cells are the strongest antigen antigenpresenting cell and only cell inducing first cellular immunoreaction. The dendritic cells migrate from a bone marrow to all organs through a blood stream. The dendritic cells collect antigen at each organ and presents the antigen to T lymphocyte at a lymphoid organ. Immature dendritic cells cannot activate T cell because the immature dendritic cells do not have cluster determinant (CD) to transfer accessory signals, such as CD40, CD54, and CD86. However, the indifferent dendritic cells can do phagocytosis and macropinocytosis for acquiring antigen to induce immunoreaction. Also, in the indifferent dendritic cells, receptors intermediating adsorptive endocytosis are well revealed at the cell wall. Such the receptors include macrophage mannose receptors similar to C lectin receptors, DEC-205(CD205), and Fc and Fc receptor. Therefore, the indifferent dendritic cells can react to the nanomolarity or picomolarity of antigen unlike other antigenpresenting cells that react to the micromolarity of antigen. In case of macrophage, eaten antigen is decomposed from lysosome to amino acid and reveals the small quantity of MHC major histocompatibility complexy-peptide combination at a cell surface. On the contrary, the dendritic cells reveal the sufficient amount of MHC-peptide combination at a cell surface by minimizing the decomposition of the eaten antigen, thereby sustaining the MHC-peptide combination in a stable condition for several days. As the dendritic cells become matured, the immunoreaction is induced. Such maturation can be controlled by various ureas. For example, bacteria, inflammatory reaction products, polysaccharides that are the cell wall component of the bacteria, IL-1, GM-CSF (granulocyte/macrophage-colony stimulating factor), and TNF (tumor necrosis factor) accelerate the maturation of the dendritic cells. The matured dendritic cells represent high concentration of a NF-B (nuclear transcription factor-B) group and a transcription factor such as Rel A/p65, Rel B, Rel C, p50, and p52. Such transcription factor controls the presentation of protein gene by participating various immunoreaction and inflammatory reaction. The signal transduction through a TNF-receptor family such as TNF-R(CD120a/b), CD40, and TRANCE/RANK (TNF-related activation induced-cytokine/receptor activator of NF-kB) activates NF-B, and stimulates the dendritic cells to induce immunoreaction. Therefore, virus or antigen participates the signal transduction of the TNF-R family or TNF-R-associated factors (TRAFs). Therefore, when leukemia cell is guided to dendritic cells and decomposed, the dendritic cells present antigen to T cell by revealing the large amount of antigen related to leukemia at a cell wall. For example, one dendritic cell activates about 100 to 3000 T cells. It was known that antileukemic T-cell responses can be induced when the clonal-expansion of each T cell is induced in a form of autocrine growth stimulation. Also, dendritic cells induced from a peripheral vascular leukemic cell of a patient having chronic myelocytic leukemia (CML) and acute myelocytic leukemia (AML) using GM-CSF, IL-4, and TNF-accelerate antileukemic T-cell proliferation and strengthen the cytotoxicity of T cell. Many researches are focused to study dendritic cells that activate T cells. However, the dendritic cells not only induce immunoreaction for external antigen but also cause the immune tolerance of a T cell, similar to using of auto antigen in a T cell.

T cell reaction is not generally induced in a tumor antigen obtained from a tumor patient. It may be because dendritic cells do not react to tumor cell. Since dendritic cells permeated to colorectal cancer or basal cell cutaneous cancer cannot present CD80 and CD86, stimulation activation for T cell becomes reduced. Therefore, the maturation of dendritic cells and the activation of cell surface molecule of the dendritic cells are absolute necessary factors to improve immunity as well as antitumor effect.

It is difficult to completely eliminate cancer or perfectly prevent cancer from metastasis using a chemotherapy that is commonly and generally used to cure a cancer patient. Also, the chemotherapy may raise serious side effects although valid dosage is given to patient. Recently, an immune chemotherapy was performed to animal models or cancer patients in order to assist the chemotherapy. Various biological response modifiers (BRM), such as cytokine controlling the biological reaction of a host and bacteria products, have been used for the immune chemotherapy. However, the immune chemotherapy has the limitation to apply to patient because gene re-combination cytokine (IL-2, IL-12, and IFN (interferon)) used to the immune chemotherapy of the cancer patient has strong toxicity and short half-life period. In order to overcome side effects of hematopoietic cell extinction when a chemotherapy or a radiotherapy is performed to cancer patients, gene recombination colony stimulating factor, granulocyte, and monocyte are partially proliferated. In order to overcome such side effects, there have been many researches in progress for developing anti-tumor nature products and for discovering folk medicine in overseas due to tumor curing effects and economical effects thereof. In Korea, various natural products known as antitumor or biological response control agent have been systemically analyzed, recently. Also, there was an attempt to develop a new anti-neoplastic drug using various polysaccharides such as lentinan, schizophyllan, and OK-432. In addition, a substance that can improve antitumor immunity by inducing antitumor cytokine to generate in a living body was discovered, and the superior antitumor effect of phospholipids such as edelfosine (ET18-OCH3) was discovered. Accordingly, there have been many researches in progress for studying phospholipids analogue.

Angelica gigas Nakai is a naturalized plant grown in Korean. Angelica gigas Nakai denotes Korean Angelica Gigantis Radix and is originally from Japan. The Angelica gigas Nakai is a perennial plant. The Angelica gigas Nakai grows up to about 60 to 90 cm, and blossoms white flower in August or September. The fruits thereof have a flat long oval shape and a narrow wing formed at edge thereof. Generally, the root of the Angelica gigas Nakai is used as medicine. It has hematinic efficacy. Therefore, the Angelica gigas Nakai has been known to be good to a person has bloodless face, frequent dizziness, and lackluster eyes and lips. Since the Angelica gigas Nakai can cure headache and control menstruation, the Angelica gigas Nakai have been frequently used for curing diseases of women after or before child birth. Also, the Angelica gigas Nakai can cure dysmenorrheal and stomachache. Furthermore, the Angelica gigas Nakai can cure constipation caused by a weak stomach, which was introduced in a book, titled colorful pictorial health guide book based on folk medicine and traditional oriental medicine by Bae, ki-hwan, pp 102-103, 2003. Since the Angelica gigas Nakai contains Coumarin derivatives such as Decursin, Decursino, and Nodakenetin, and refined oil components such as α-Pinene, Limonene, β-Eudesmol, and Elemol, the saccharide can control uterus function, damper down, perform antibacterial action, perform diarrhea action, and cure the shortage of vitamin E. Therefore, the Angelica gigas Nakai is used as medicine for anemia, pain, coelenteron, emmenagogue, and gynaecopathy. Also, Angelica gigas Nakai may be eaten as herbs in a spring time.

According to the related research about Angelica gigas Nakai, it was known that ether extracted from the Angelica gigas Nakai excites an intestinal tract and a womb extracted from a rabbit and also accelerates a blood pressure and breathing. Meanwhile, isolated decursin and decursion1 were identified to paralyze the intestinal tract extracted from a rabbit. The isolated decursin and decursion1 also restraint a heart extracted from a flog, restraint the breathing of a rabbit, and reduces a blood pressure of a rabbit. Furthermore, it was known that the isolated decursin and decursion1 restraint mice's spontaneous movement (Korean Journal of Pharmacognosy, volume 1, pp 25-32, 1970).

The immunity control activity and the anticancer activity of polysaccharides separated from Angelica gigas Nakai have been introduced through articles and patents as follows.

1. Immunopharmacol 40, 39-48, 1998

2. Immunopharmacol 43, 1-9, 1999

3. Journal of Pharmacolology and Experimental Therapeutics 294(2), 548-554, 2000

4. Immunopharmacol 49(3), 275-284, 2000

5. Immunopharmacol 1(2), 237-245, 2001

6. Korea Patent No. 0252194

7. Korea Patent No. 0441644

8. Korea Patent Application No. 2005-90623

It was known that angelan activates the immunity of a macrophage and a B cell, and it means that angelan has anticancer effect. It was proved that the angelan activate the B cell and the macrophage by combining to a cell molecule receptor such as toll-like receptor4 and CR3 and activating NF-kB and mitogen-activated protein kinases such as ERK, JNK, and p38. Furthermore, it was known that the angelan has the anticancer effect by restraining the attachment and migration of cancer cells.

However, it is not known that the angelan, polysaccharides separated from Angelica gigas Nakai, activates dendritic cells.

### DISCLOSURE OF INVENTION

### Technical Problem

It is an object of the present invention to provide the use of Angelica gigas Nakai as an immunomodulating drug by researching the effects of dendritic cells of angelan which is polysaccharides decomposed from Angelica gigas Nakai.

### Technical Solution

In accordance with an aspect of the present invention, an immunomodulating drug composition improves immunologic function of dendritic cells that contain about 0.1 to 50 weight % of angelan that is polysaccharides from Angelica gigas Nakai based on the total weight of a composition, a carrier, an excipient, and a diluent, which are pharmaceutically allowed.

A pharmaceutical composition having polysaccharides according to an embodiment of the present invention may further include predetermined carrier, diluent, or excipient, which is generally used for preparing a pharmaceutical composition.

A pharmaceutical composition according to an embodiment of the present invention may be prescribed through a pharmaceutically allowed form thereof. The pharmaceutical composition may be independently prescribed, or may be prescribed with another pharmaceutical composition.

A pharmaceutical composition according to an embodiment of the present invention may be manufactured in various forms of medicine including an oral administration drug such as powders, granulations, tablets, capsules, emulsion, and aerosol, external preparation, pessary, and sterile injection liquid. A carrier, a diluent, and an excipient, which can be included in the composition including extract or compound, may be lactose, dextrose, sucrose, sorbitol, mannitol, xylitol, erythritol, maltitol, starch, acacia gurn, alginate, gelatin, calcium phosphate, calcium celkate, cellulose, methyl cellulose, crystalline cellulose, polyvinyl pyrolidone, water, methyl hydroxy benzoate, propyl hydroxy benzoate, talc, magnesium sterate, and minerals. A diluent or an excipient such as a filling agent, an extender, a bonding agent, a wetting agent, a solution agent, and a surface active agent, are used to manufacture medicine. Solid medicine for oral administration includes tablets, pills, granulations, and capsules. Such a solid medicine is manufactured by mixing the extract or the compound with at least one of excipients including starch, calcium carbonate, sucrose, lactose, and gelatin. Beside the simple excipient, a lubricant such as magnesium sterate, and talc is also used. Liquid medicine for oral administration includes suspensions, emulsions, and syrup as well as simple diluent such as water and liquid paraffin. The liquid medicine may include various excipients including a wetting agent, an aromatic, and embalmment. Medicine for parenteral administration includes sterile injection solution, non-watery solvent, suspension, emulsion, lyophilization agent and pessary. As the non-watery solvent and suspension, propylene glycol, poly ethylene glycol, vegetable oil such as olive oil, and ester such as ethyl oleate may be used. As the pessary, witepsol, macrogol, tween 61, cacao butter, and glycerol gelatin may be used.

The polysaccharides can be injected into a mammal such as mouse, rat, livestock, and human through various paths. All of injection methods may be used, such as through a mouth, a rectum, a vein, a muscle, a skin, an epidural in a womb, or intracerebroventricular injection.

Since the polysaccharides do not have toxicity and side effects, it can be used for long time with relief.

### Advantageous Effects

The present invention relates to a pharmacological composition for activating dendritic cells having polysaccharides from Angelica gigas Nakai. Angelan, polysaccharides separated from Angelica gigas Nakai, can increase the revelation of surface molecule of dendritic cells, the secret of cytokine of dendritic cells, the proliferation capability of T cells of dendritic cells, and the generation of cytokine such as IL-2.

### BRIEF DESCRIPTION OF THE DRAWINGS

The invention will be described in detail with reference to the following drawings in which like numerals refer to like elements.

FIG. 1 is a graph illustrating the phenotype of dendritic cells with angelan processed;

FIG. 2 is a graph illustrating the antigen uptake degree of dendritic cells with angelan processed;

FIG. 3 is a diagram illustrating the cytokine secrete of dendritic cells with angelan processed;

FIG. 4 is a diagram illustrating the signal transduction variation of dendritic cells with angelan processed; and

FIG. 5 is a diagram illustrating the T cell stimulation effect of dendritic cells with angelan processed.

### BEST MODE FOR CARRYING OUT THE INVENTION

Preferred embodiments of the present invention will be described in a more detailed manner with reference to the drawings.

### First Embodiment: Separate Polysaccharides from Angelica gigas Nakai and Analyze Composition Thereof.

Angelan, polysaccharides from Angelica gigas Nakai, was separated using a method introduced in an article in Immunophrmacology 40, p 39 to 48, 1998. The root of Angelica gigas Nakai cut into small pieces. The small pieces of the root were putted in a hot water for one hour. Then, a filtrate was collected by filtering the hot water with the root pieces through the four layers of gauzes and a filter paper. Ethanol as much as three times of the collected filtrate was mixed with the collected filtrate. The collected filtrate with the ethanol mixed had been left for three hours at 4° C. Then, brown macromolecule fractionations were collected by centrifuging extracted precipitate.

The macromolecule fractionations can be easily collected through precipitation using a small amount of ethanol. Although the macromolecule fractionations were melted and boiled for twenty minutes, precipitation was not formed by denaturalized protein. Therefore, it was expected that the fractionations contain a large amount of non-protein macromolecule substance.

If the fractionations were absorbed using DEAE-cellulose that is a negative ion exchange resin, it was observed that the most of colored substances were absorbed at the resin. Two acid and neutral fractionations were prepared using DEAE-cellulose absorption. The fractionations were identified as polysaccharides containing a small amount of protein, and acid polysaccharide among the polysaccharides was named as angelan.

If the composition of the angelan is analyzed, the angelan contains about 85% to 90% of saccharide, about 7% to 8% of protein, and about 15.5% to 68% of uronic acid. The angelan also contains a comparatively large amount of calcium ion and magnesium ion, and a small amount of iron, aluminum, manganese, kalium, natrium, phosphorus, and sulfur.

In order to analyze saccharide composition, a purified sample is hydrolyzed with 2M triple flow acetic acid, and the polysaccharide is analyzed using a thin layer liquid chromatography (TLC) and ion exchange high speed liquid chromatography (HPLC).

According to the result of analysis, the polysaccharide contains the large amount of galaturonic acid, galactose, and arabinose, and a small amount of mannose, rhamnose, and xylose.

### First Production Example: Prepare Animal for Test

C57BL/6 female mouse (Dae-han BioLink, chungbuk) each having a weight of about 18 to 22 g were used as an animal for test. The mouse were allowed to freely have food and water at constant condition (temperature: 21±2° C., light and shade: 12 hours of light and shade cycle) in a breeding room. The mouse had been purified in the breeding room for seven days before a test starts.

### Second Production Example: Prepare Dendritic Cells

Bone marrow cells were separated from the C57BL/6 mouse. Then, the bone marrow cells were cultivated at the concentration of about 1×10⁶ cell/□. The bone marrow cells had been cultivated for eight days at the concentration of about 2 ng/□ by adding GM-CSF. Dendritic cells generated while cultivating were known as immature cell. Then, angelan is added to the immature dendritic cells at the concentration of 10˜100□/□, and the immature dendritic cells had been additionally cultivated for 48 hours. Lipopolysaccharide was added at the concentration of □/□.

### MODE FOR THE INVENTION

### Second Embodiment: Influence of Angelan, Polysaccharide from Angelica gigas Nakai, to dendritic cells

In order to analyze the influence of the angelan separated in the first embodiment to dendritic cells, the revelation of surface molecules of dendritic cells, the secrete of cytokine of dendritic cells, the proliferation capability of T cell of dendritic cells, and the generation of cytokine such as IL-2 from T cell were researched.

### First Experimental Example: Influence to Cell Surface Molecule of Dendritic Cells

Dendritic cells were prepared using the same method of the second production example. After the dendritic cells were cultivated for ten days, the dendritic cells were collected. The collected dendritic cells were dyed with antibody such as FITC(Fluorescein Isothiocyanate)-CD11c, PE(R-Phycoerythrin)-CD80, PE-CD86, PE-, PE-MHC II. Then, the dyed dendritic cells were analyzed using a flow cytometer, and the analysis result was expressed as Mean Fluorescence Intensity (MFI). The higher MFI value denotes strong revelation of surface molecule and means that the maturation degree of the dendritic cells increases. CD11c denotes the surface molecule selectively revealed in the dendritic cells, and CD80, CD86, and MHC-II are surface molecules that are more revealed as the dendritic cells become more maturated.

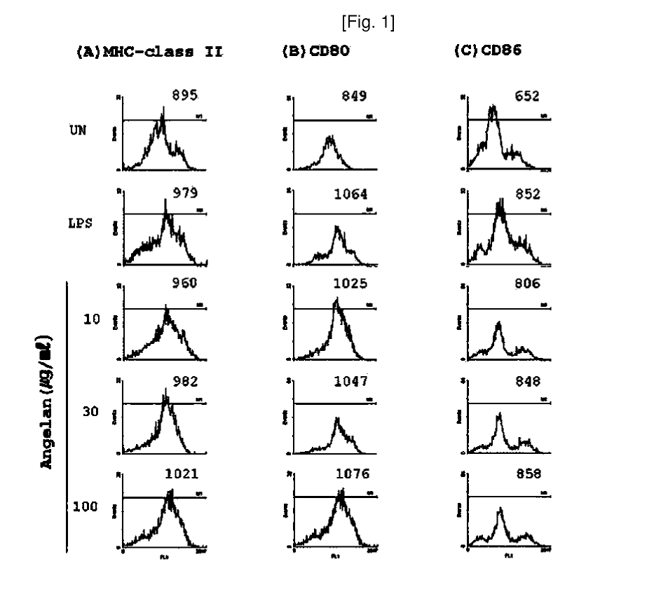

As shown in FIG. 1, the expressivity of MHC-II is about 895 in case of a control group. The expressivity of MHC-II increases to 979 in a LPS treated group. Also, the expressivity of MHC-II abruptly increases to 960, 982, and 1021 in an angelan treated group according to the concentration thereof. The expressivity of CD80 is 849 in a control group. The expressivity of CD80 increases to 1064 in a LPS treated group. Also, the expressivity of CD80 abruptly increases to 1025, 1047, and 1076 according to the concentration thereof. The expressivity of CD86 is 652 in a control group. The expressivity of CD86 increases to 852 in a LPS treated group. Also, the expressivity of CD86 abruptly increases to 806, 848, and 856 according to the concentration thereof.

### Second Experimental Example: Influence to Antigen Uptake of Dendritic Cells

Although immature dendritic cells have superior antigen uptake capability, it was known that the antigen uptake capability of mature dendritic cells decreases. If the angelan increases the maturity of the dendritic cells, it is expected that the antigen uptake capability would decrease. In order to prove the expectation, a following experiment was performed.

Dendritic cells were prepared using the same method of the second production example. After the dendritic cells were cultivated for ten day, the dendritic cells were collected. Then, the dendritic cells were processed using the antigen of FITC-dextran at the concentration of 0.7 mg/□ for two hours. After remained FITC-dextran was completely removed by cleaning the dendritic cells, the amount of antigen in the dendritic cells was measured using a flow cytometry. The dendritic cells were dual-dyed using the antibody of CD11c-PE, which is selectively connected to the dendritic cells, in order to analyze an antigen uptake degree after the dendritic cells were selectively separated.

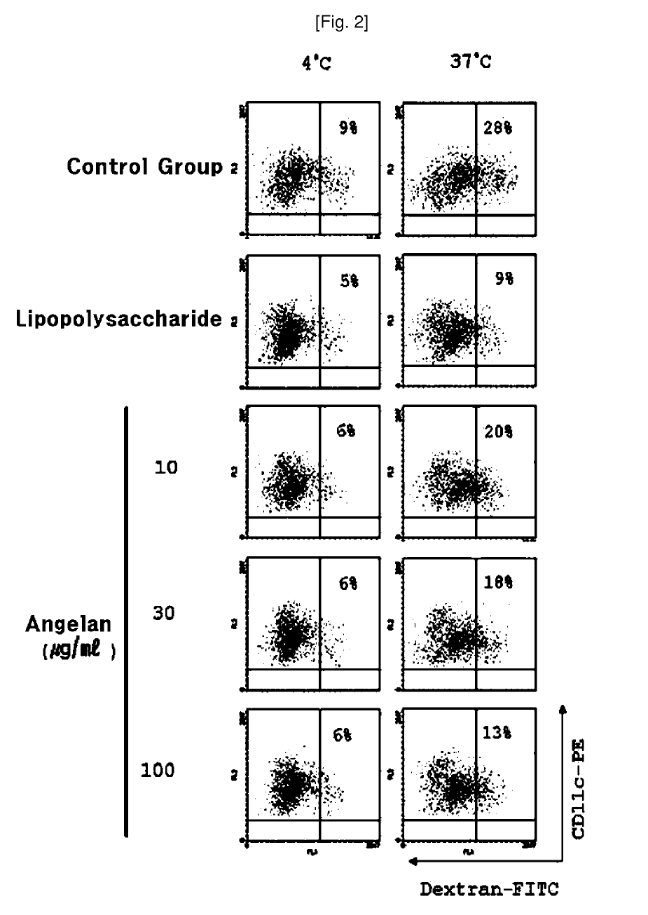

As shown in FIG. 2, the antigen uptake degree immature dendritic cells (control group) generated while the dendritic cells were cultivated for two hours at 37° C. was about 28%. The antigen uptake degree of dendritic cells with lipopolysaccharide processed decreases to about 9%. The antigen uptake degree of dendritic cells with angelan processed decreases to about 20%, 28%, and 13%. It means that the maturity of the dendritic cells increases by the LPS process and the angelan process. When the dendritic cells are cultivated at 4° C., the antigen uptake is completely stopped. Therefore, all cells have low antigen uptake degree, for example, about 5 to 9%. It means immunoreaction where the antigen uptake actively occurs.

### Third Experimental Example: Influence to Generation of Cytokine of Dendritic Cells

The important characteristic of mature dendritic cells is the secret of cytokine. Especially, it was known that the secret of IL-12 is very important to active T cell.

Dendritic cells were prepared using the same method of the second production example. After the dendritic cells had been cultivated for ten days, the amount of cytokine secreted from the dendritic cells was measured using an enzyme-linked immunoassay method. Materials for experiments were purchased from a company, R & D systems, and the experiment was performed based on an experiment rule provided from the company R & D systems.

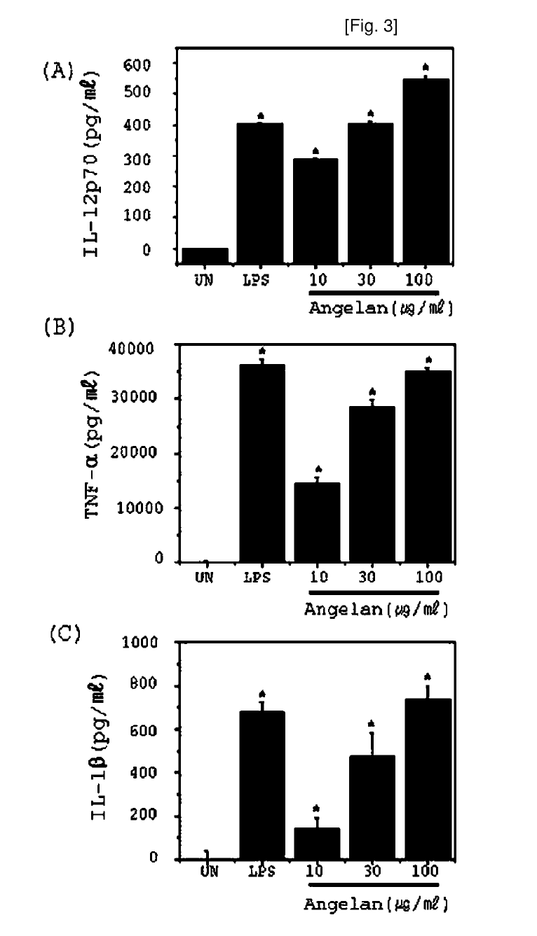

As shown in FIG. 3, although immature dendritic cells (UN, control group) cannot generate cytokine such as IL-12, TNF-α, and IL-1b, the generation of cytokine strongly increases in the dendritic cells with LPS or angelan processed. It means that the dendritic cells become matured by the angelan process. Accordingly, the secrete of cytokine increases.

### Fourth Experimental Example: Influence to Signal Transduction of Dendritic Cells

It is known that mitogen-activated protein kinases such as ERK, JNK, and p38 become activated while the dendritic cells are maturating. After the immature dendritic cells were created using the same method of the second production example, LPS and angelan were processed for fifteen minutes, and all of proteins were separated by destroying cells. The protein amount of phosphorylated ERK, JNK, and p38 was measured using a western bullating method using a predetermined antibody.

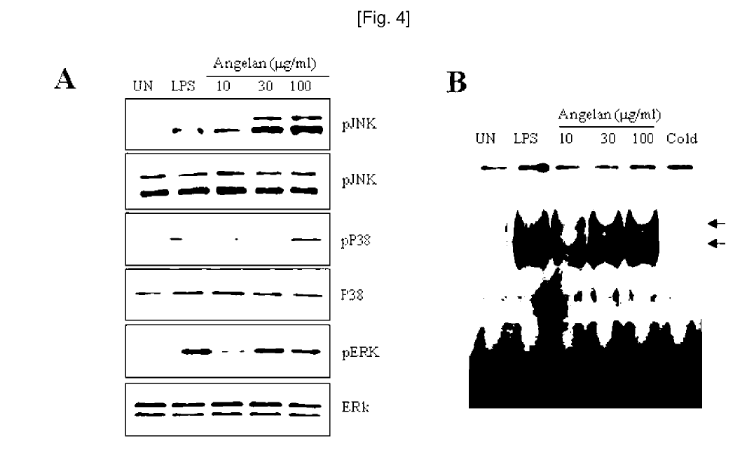

As shown in FIG. 4A, the amount of phosphorylated p-ERK, p-JNK, and p-p38 increases by LPS and angelan process.

It was known that NF-κB(nuclear factor-kappa B) becomes activated while the dendritic cells were matured. After immature dendritic cells are created using the same method of the second production example, LPS and angelan had been processed for six hours. All protein is separated by separating a cell nucleus and destroying the separated cell nucleus. The amount of NF-κB in the cell nucleus is measured using an electromobility shift assay.

As shown in FIG. 4B, the amount of NF-κB increases.

### Fifth Experimental Example: Influence to Immunologic Function

The most important immunologic function in mature dendritic cells is to activate T cell. The immature dendritic cells uptakes antigen, is matured, moves to an immune organ having T cells, and activate the T cells. The activated T cells are proliferated and secrete cytokine, actively.

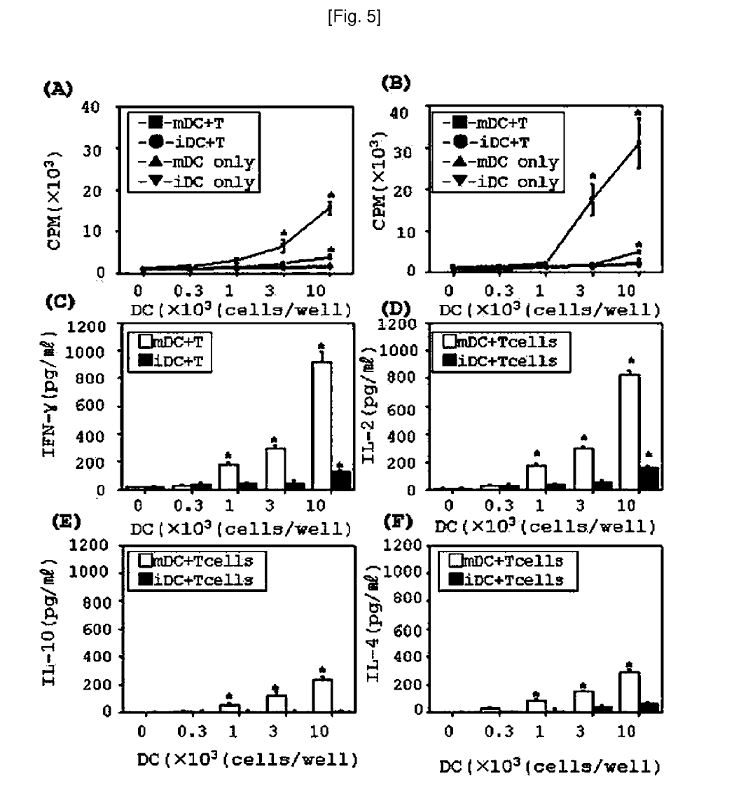

After mature dendritic cells (mDC) were created by processing angelan on the immature dendritic cells (iDC), the mature dendritic cells were cultivated for three days as shown in FIG. 5A or for five days as shown in FIG. 5B with allogeneic T cells. T cells are proliferated by processing [³H]-thymidine for last sixteen hours of cell cultivation. Then, the cell proliferation is measured by flowing radioactivity material into the DNA of T cell.

As shown in FIG. 5A and FIG. 5B, mature dendritic cells (mDC) strongly increases the proliferation of T cell.

Also, the amount of cytokine such as IFN-g, IL-2, IL-10, and IL-4, secreted from T cell was measured. The amount of cytokine abruptly increases in the T cell commonly cultivated with the dendritic cells. However, the amount of cytokine, generated in the T cell commonly cultivated with immature dendritic cells, is very small.

It means that the dendritic cells with angelan processed are the mature dendritic cells having a superior immunologic function that activates T cells.

### INDUSTRIAL APPLICABILITY

The angelan, polysaccharides separated from Angelica gigas Nakai, can improve the revelation of surface molecule of dendritic cells, the secret of cytokine of dendritic cells, the proliferation capability of T cells of dendritic cells, and the generation of cytokine such as IL-2. Therefore, the present invention is very useful in medical industry.

1. A immunomodulating drug composition for improving an immunologic function of dendritic cells that contain about 0.1 to 50 weight % of angelan, which is polysaccharides from Angelica gigas Nakai, for a total weight of a composition, and pharmaceutically allowed carrier, diluent and excipient.
